(19) United States
(12) Patent Application Publication
(10) Pub. No.: US 2026/0035621 A1
(43) Pub. Date: Feb. 5, 2026

(54) METHOD FOR EXTRACTION AND ANALYSIS OF POLYCYCLIC AROMATIC HYDROCARBONS IN PETROLEUM PRODUCTS

(71) Applicant: HollyFrontier LSP Brand Strategies LLC, Dallas, TX (US)

(72) Inventors: Robert Ingraham, Dallas, TX (US); Matthew Mason, Dallas, TX (US)

(73) Assignee: HollyFrontier LSP Brand Strategies LLC, Dallas, TX (US)

(21) Appl. No.: 19/275,007

(22) Filed: Jul. 21, 2025

Related U.S. Application Data
(60) Provisional application No. 63/678,140, filed on Aug. 1, 2024.

(51) Int. Cl. C10G 21/20; C10G 21/02; C10G 21/14; C10G 21/16; G01N 30/06; G01N 33/28

(52) U.S. Cl. CPC C10G 21/20; C10G 21/02; C10G 21/14; C10G 21/16; G01N 30/06; G01N 33/28; C10G 2400/30; G01N 2030/062

(57) ABSTRACT

A method for extraction and analysis of polycyclic aromatic hydrocarbons (PAHs) uses a solvent mixture that is effective for extraction and also compatible with disposable polypropylene solid phase extraction (SPE) tubes. The petroleum product is mixed with an organic solvent, then an extraction solvent made up of a volume ratio of 70:30 to 90:10 acetonitrile to ethyl acetate is added to the mixture. The organic solvent and PAHs are adsorbed in the extraction solvent mixture, leaving the oils behind. The extract, including the solvents and PAHs, can be transferred to a disposable SPE tube for further sample processing, analysis and disposal.

This application claims priority to U.S. Provisional Patent Application Ser. No. 63/678,140, entitled “Method for Extraction and Analysis of Polycyclic Aromatic Hydrocarbons in Petroleum Products,” filed Aug. 1, 2024, the entire contents of which are incorporated by reference herein.

### BACKGROUND

The present disclosure relates to methods and compositions for the extraction and analysis of polycyclic aromatic hydrocarbons (PAHs) found in petroleum-based products.

Industry standard practices for the extraction of PAHs from petroleum-based oils are time-consuming, with low sample through-put, and are not compatible with the use of polypropylene solid phase extraction (SPE) tubes. The use of SPE tubes and cartridges would introduce an efficiency and reduced environmental impact to the PAH extraction and clean-up process. However, the organic solvents pentane and cyclohexane typically used to extract PAHs from petroleum-based oils cannot be used with the polypropylene tubes because these solvents will degrade the polypropylene tubes. Certain SPE cartridges are designed for conducting PAH extractions in vegetable oils, but this method is not robust enough for the extraction of PAHs from certain petroleum products, including but not limited to precursor oils, extender oils, asphalt pitch, and crude oil. Existing practices for extraction of PAHs from petroleum may also utilize harmful chemicals such as dichloromethane, may require small sample sizes and may take an extended period of time.

What is needed is a method for extraction of PAHs from petroleum products that is compatible with disposable SPE tubes and involves less harmful chemicals, allowing for a more efficient and environmentally friendly process.

### SUMMARY

The present disclosure pertains to extraction of polycyclic aromatic hydrocarbons (PAHs) from petroleum-based products.

The present composition and method for extraction and analysis of polycyclic aromatic hydrocarbons (PAHs) involves a solvent mixture that is effective for extraction and also compatible with disposable polypropylene solid phase extraction (SPE) tubes. First, the petroleum product is mixed with an organic solvent such as pentane to fully dissolve the sample. Internal standards are added, and the sample is vortex mixed until the oil is fully dissolved. Then an extraction solvent mixture made up of a volume ratio of 70:30 to 90:10 acetonitrile to ethyl acetate is added. The organic solvent and PAHs are adsorbed into the extraction solvent mixture, leaving the oils behind. Subsequently, the extract, including the solvent mixture and PAHs, can be transferred to a disposable SPE tube for sample clean-up prior to analysis using GC Mass Spectrometry, and responsible disposal.

Advantages of the disclosed composition and method include reduced extraction times, increased productivity, and elimination of the need for hazardous solvents such as dichloromethane. While an industry standard practice method requiring an overnight preparation step could extract 2 samples on the second day over the course of 8+ hours, the present composition and method has the potential to extract 5 samples in duplicate with additional quality control (QC) samples over the course of 4 hours. The composition and method described herein allow for the extraction of larger size samples (500 mg vs. standard practice 70 mg). This allows for a higher analyte extraction and recovery resulting in a more robust response to the further analysis. The method could also be scaled up to produce process scale benefits, i.e. effective extraction of PAHs and other aromatic compounds from production petroleum oils, not just extraction of small samples to aid in analysis of the PAHs.

### BRIEF DESCRIPTION OF THE DRAWINGS

FIG. 1 shows a photograph of a series of oil samples mixed with an 80:20 acetonitrile—ethyl acetate solvent mixture intended for use in analyzing PAHs in vegetable oils.

FIG. 2 shows a photograph of a series of precursor oil samples treated with preferred embodiments of the solvent mixture described herein.

FIG. 3 shows a photograph of the same samples as in FIG. 2 after the samples were centrifuged to separate the oil from the extraction mixture.

FIG. 4 shows GC Mass Spectrometry peaks for certain exemplary compounds identified from an analysis of an extract prepared from a petroleum product using preferred embodiments of the methods and solvent mixture described herein.

FIG. 5 shows the PAH quantification from Sundex 8000EU after a series of extractions using preferred embodiments of the methods described herein and using the industry standard.

### DETAILED DESCRIPTION OF PREFERRED EMBODIMENTS

The present disclosure relates to the use of a solvent mixture to prepare an extract of polycyclic aromatic hydrocarbons (PAHs) from petroleum-based products, where the solvent mixture that is used allows for further sample processing of the PAHs in disposable polypropylene solid phase extraction (SPE) tubes.

In preferred embodiments, the petroleum-based product is first mixed with and dissolved in an organic solvent such as any pentane, hexane, heptane, or octane. The mixture is vortexed until the oil is fully dissolved. After the dissolution in the organic solvent, internal standard that consists of deuterated or ¹³C labeled PAH standards is added and the mixture is vortexed again. The petroleum solvent solution is then mixed with a solvent mixture of acetonitrile and ethyl acetate. In preferred embodiments, the solvent mixture has a volume/volume ratio of 70:30 to 90:10 of acetonitrile and ethyl acetate, preferably an 80:20 solvent mixture (volume/volume) of acetonitrile and ethyl acetate. The acetonitrile—ethyl acetate extraction solvent ratio can vary as long as organic solvent remains full miscible.

The eight PAHs typically identified are benzo[a]anthracene, chrysene, benzo(b)fluoranthene, benzo(j)fluoranthene, benzo(k)fluoranthene, benzo(e)pyrene, benzo(a)pyrene, and dibenzo(a,h)anthracene.

Table 1 below lists examples of organic solvents (pentane, isooctane, heptane, cyclohexane) that are fully miscible in eight mL of an acetonitrile ethyl acetate solvent mixture (80/20, 90/10, 70/30) and shows the results of using the method described herein to extract PAHs from an exemplary petroleum product (Sundex 8000 EU oil) using the listed Solvent Mixture. In the examples below, 500 mg of the petroleum-based product is mixed with and dissolved in 1 mL of an exemplary organic solvent. The organic solvent and extracted PAHs are adsorbed into the acetonitrile-ethyl acetate solvent mixture, forming a single-phase extraction mixture and leaving the petroleum oil residue behind.

TABLE 1

| | PAH Analysis* (ppm) | | | | | | | | |
|---|---|---|---|---|---|---|---|---|---|
| Solvent Mixture | 1 | 2 | 3 | 4 | 5 | 6 | 7 | 8 | T |
| 918-A 1417525 | 0.098 | 1.018 | 0.777 | 0.140 | 0.015 | 3.720 | 0.294 | 0.245 | 6.307 |
| Pentane 80/20 | | | | | | | | | |
| May 23, 2025 | | | | | | | | | |
| 918-B 1417525 | 0.108 | 0.985 | 0.767 | 0.135 | 0.015 | 3.344 | 0.402 | 0.250 | 6.006 |
| Pentane 80/20 | | | | | | | | | |
| May 23, 2025 | | | | | | | | | |
| 918-A 1417525 | 0.113 | 1.087 | 0.743 | 0.131 | 0.019 | 3.599 | 0.425 | 0.297 | 6.414 |
| Pentane 90/10 | | | | | | | | | |
| May 23, 2025 | | | | | | | | | |
| 918-B 1417525 | 0.102 | 1.003 | 0.738 | 0.160 | 0.023 | 3.593 | 0.362 | 0.262 | 6.242 |
| Pentane 90/10 | | | | | | | | | |
| May 23, 2025 | | | | | | | | | |
| 918-A 1417525 - | 0.099 | 1.106 | 0.802 | 0.175 | 0.018 | 3.866 | 0.367 | 0.292 | 6.725 |
| Isooctane 80/20 | | | | | | | | | |
| May 9, 2025 | | | | | | | | | |
| 918-B 1417525 - | 0.115 | 1.023 | 0.894 | 0.184 | 0.026 | 3.911 | 0.384 | 0.315 | 6.852 |
| Isooctane 80/20 | | | | | | | | | |
| May 9, 2025 | | | | | | | | | |
| 918-A 1417525 - | 0.112 | 1.224 | 0.830 | 0.178 | 0.022 | 3.826 | 0.402 | 0.323 | 6.917 |
| Heptane 80/20 | | | | | | | | | |
| May 9, 2025 | | | | | | | | | |
| 918-B 1417525 - | 0.121 | 1.091 | 0.838 | 0.181 | 0.025 | 3.742 | 0.541 | 0.371 | 6.909 |
| Heptane 80/20 | | | | | | | | | |
| May 9, 2025 | | | | | | | | | |
| 918-A 1417525 - | 0.105 | 0.923 | 0.813 | 0.144 | 0.019 | 3.837 | 0.364 | 0.291 | 6.496 |
| Cyclohexane 80/20 | | | | | | | | | |
| May 12, 2025 | | | | | | | | | |
| 918-B 1417525 - | 0.090 | 0.990 | 0.823 | 0.174 | 0.018 | 3.883 | 0.393 | 0.297 | 6.668 |
| Cyclohexane 80/20 | | | | | | | | | |
| May 12, 2025 | | | | | | | | | |
| 918-A 1417525 - | 0.096 | 0.884 | 0.838 | 0.135 | 0.015 | 3.745 | 0.292 | 0.250 | 6.255 |
| Pentane 70/30 | | | | | | | | | |
| May 15, 2025 | | | | | | | | | |
| 918-B 1417525 - | 0.086 | 1.009 | 1.135 | 0.168 | 0.009 | 3.770 | 0.263 | 0.276 | 6.716 |
| Pentane 70/30 | | | | | | | | | |
| May 15, 2025 | | | | | | | | | |
| 918-A 1417525 - | 0.090 | 1.077 | 1.173 | 0.156 | 0.015 | 3.913 | 0.351 | 0.302 | 7.076 |
| Cyclohexane 70/30 | | | | | | | | | |
| May 15, 2025 | | | | | | | | | |
| 918-B 1417525 - | 0.089 | 1.026 | 0.804 | 0.148 | 0.018 | 3.883 | 0.385 | 0.303 | 6.656 |
| Cyclohexane 70/30 | | | | | | | | | |
| May 15, 2025 | | | | | | | | | |
| 918-A 1417525 - | 0.103 | 1.108 | 0.860 | 0.150 | 0.015 | 3.879 | 0.407 | 0.297 | 6.818 |
| Heptane 70/30 | | | | | | | | | |
| May 19, 2025 | | | | | | | | | |
| 918-B 1417525 - | 0.095 | 0.987 | 0.821 | 0.155 | 0.018 | 3.815 | 0.330 | 0.313 | 6.535 |
| Heptane 70/30 | | | | | | | | | |
| May 19, 2025 | | | | | | | | | |
| 918-A 1417525 - | 0.094 | 1.087 | 1.183 | 0.151 | 0.018 | 3.934 | 0.397 | 0.314 | 7.178 |
| Isooctane 70/30 | | | | | | | | | |
| May 19, 2025 | | | | | | | | | |
| 918-B 1417525 - | 0.085 | 0.999 | 0.743 | 0.161 | 0.017 | 3.753 | 0.301 | 0.272 | 6.331 |
| Isooctane 70/30 | | | | | | | | | |
| May 19, 2025 | | | | | | | | | |
| Average ppm | 0.100 | 1.035 | 0.866 | 0.157 | 0.018 | 3.778 | 0.370 | 0.293 | 6.617 |
| 99.9% Confidence | 0.008 | 0.060 | 0.111 | 0.013 | 0.003 | 0.114 | 0.049 | 0.024 | 0.246 |
| Level+ | | | | | | | | | |

*1 = Benz(a)anthracene, 2 = Chrysene, 3 = Benzo(b)fluoranthene, 4 = Benzo(k)fluoranthene, 5 = Benzo(j)fluoranthene, 6 = Benz[e]pyrene, 7 = Benz[a]pyrene, 8 = Dibenz(a,h)anthracene, T = Total EU8 PAHs

The extract prepared according to the preferred methods described herein, containing a mixture of PAHs and solvent, may be transferred into a SPE tube or cartridge for sample clean-up prior to further analysis, including mass spectrometry (MS) or gas chromatography—mass spectrometry (GC-MS), and ease of responsible disposal.

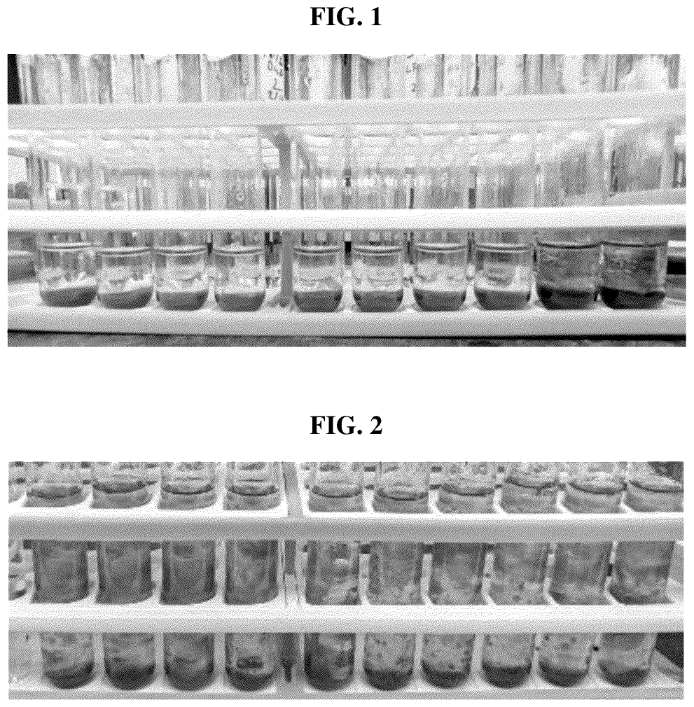

FIG. 1 shows a photograph of a series of oil samples mixed with an 80:20 acetonitrile—ethyl acetate solvent mixture intended for use in analyzing PAHs in vegetable oils. There is poor mixing of oil with the extraction solvent in the first eight samples from the left—i.e., the solvent layer is clear. These eight samples are precursor oils. There is improved mixing of oil with the extraction solvent in the last two samples on the right—i.e., the solvent layer contains oil. These last two samples are extender oils, not precursor oils. The addition of an organic solvent is considered necessary to achieve adequate extraction of PAHs from all petroleum products tested.

FIG. 2 shows a photograph of a series of precursor oil samples treated with the solvent mixture described herein. First, a 500 mg oil sample was dissolved in 1 mL of pentane. Then, 8 ml of an 80:20 acetonitrile-ethyl acetate mixture was added to this pentane oil solution and vortex mixed. The excellent mixing of oil with the extraction solvent can be seen in all of the samples of precursor oil in FIG. 2. In each sample there is oil in the solvent layer.

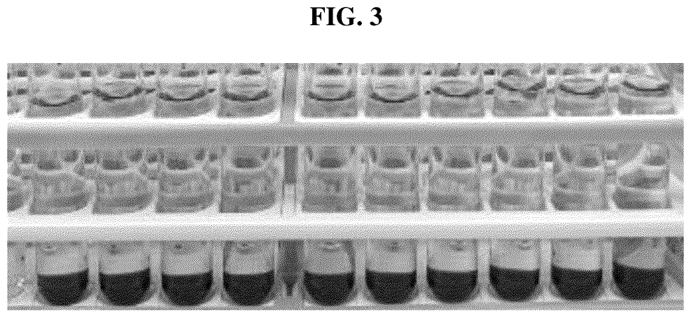

FIG. 3 shows a photograph of the same samples as in FIG. 2 after the samples were centrifuged to separate the oil from the extraction mixture. The pentane and PAHs are fully absorbed into the acetonitrile—ethyl acetate mixture. The oil residue remains at the bottom of each tube.

The use of the preferred embodiments of the solvent mixture described herein allows the extract (containing the PAHs) to be processed using polypropylene SPE tubes. This provides an “off-the-shelf” and ready to go sample clean-up method. The extraction method described herein also leads to increased PAH sample analysis output. In certain examples, the analysis changes from two samples in eight hours with no quality control to five samples in four hours with quality control. In certain embodiments, the final extract is blown dry and reconstituted in 0.5 mL of toluene prior to GC-MS/MS analysis.

The solvent mixture and method described herein can be used in connection with all petroleum products, including but not limited to refined aromatic oils, refined paraffinic oils, crude oil distillation cuts, crude oil, and asphalt pitch.

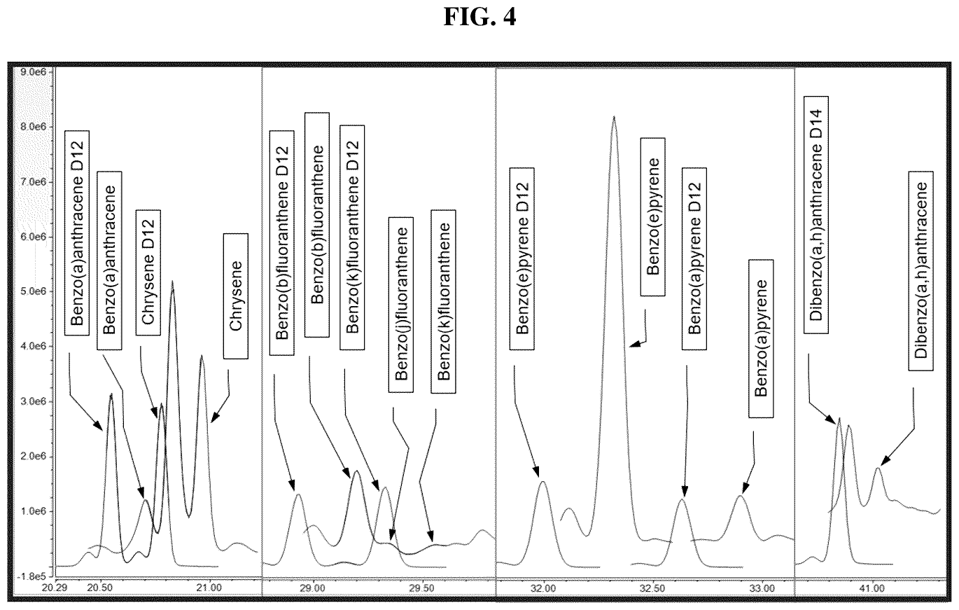

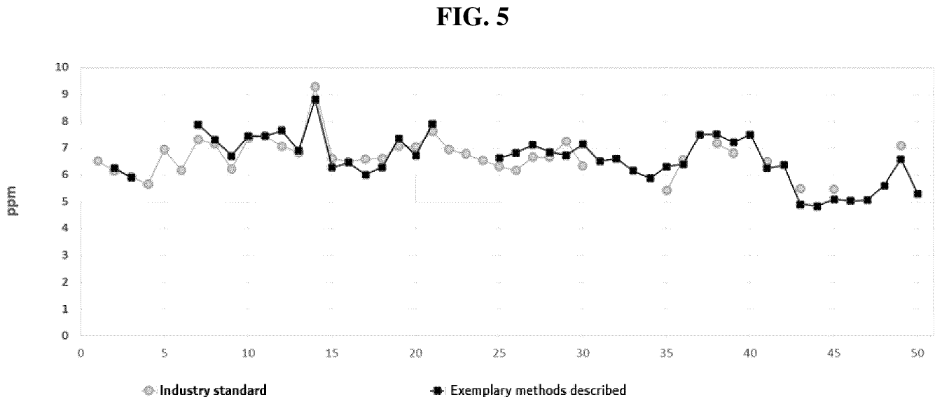

GC Mass spectrometry analysis of extracts obtained using preferred embodiments of the method and solvent mixture described herein produce clear, quantifiable PAH peaks with clean background. FIG. 4 shows peaks for certain exemplary compounds identified from an analysis of an extract prepared from a petroleum product using the methods and solvent mixture described herein. FIG. 5 shows the results of a series of extractions using the industry standard analysis and the methods described herein, demonstrating the repeatability of PAH extraction and quantification from petroleum products using the current methods.

Tables 2 and 3 below show a comparison between the total amount of eight (8) PAHs, typically identified in industry standard analysis, that were identified by using an industry standard and extracted from an exemplary petroleum product in Table 2 (Sundex 8000 EU oil) and a series of distillate aromatic extracts (DAE) in Table 3 by using the exemplary method and solvent mixture described herein.

TABLE 2

| | Total (8) PAH (ppm) | | |
|---|---|---|---|
| | Industry | Exemplary Extraction Process | |
| Sample | Standard | Described Herein | RSD |
| 1 - 8000 EU | 6.53 | | |
| 2 - 8000 EU | 6.16 | 6.26 | 1.6% |
| 3 - 8000 EU | 5.94 | 5.90 | 0.7% |
| 4 - 8000 EU | 5.65 | | |
| 5 - 8000 EU | 6.94 | | |
| 6 - 8000 EU | 6.18 | | |
| 7 - 8000 EU | 7.32 | 7.88 | 7.4% |
| 8 - 8000 EU | 7.16 | 7.32 | 2.2% |
| 9 - 8000 EU | 6.22 | 6.71 | 7.6% |
| 10 - 8000 EU | 7.36 | 7.46 | 1.3% |
| 11 - 8000 EU | 7.46 | 7.44 | 0.3% |
| 12 - 8000 EU | 7.07 | 7.65 | 7.9% |
| 13 - 8000 EU | 6.82 | 6.90 | 1.2% |
| 14 - 8000 EU | 9.30 | 8.81 | 5.4% |
| 15 - 8000 EU | 6.61 | 6.27 | 5.3% |
| 16 - 8000 EU | 6.51 | 6.45 | 0.9% |
| 17 - 8000 EU | 6.60 | 6.00 | 9.5% |
| 18 - 8000 EU | 6.62 | 6.28 | 5.3% |
| 19 - 8000 EU | 7.06 | 7.36 | 4.2% |
| 20 - 8000 EU | 7.05 | 6.72 | 4.8% |
| 21 - 8000 EU | 7.62 | 7.90 | 3.6% |
| 22 - 8000 EU | 6.95 | | |
| 23 - 8000 EU | 6.77 | | |
| 24 - 8000 EU | 6.55 | | |
| 25 - 8000 EU | 6.31 | 6.62 | 4.8% |
| 26 - 8000 EU | 6.18 | 6.82 | 9.8% |
| 27 - 8000 EU | 6.66 | 7.12 | 6.7% |
| 28 - 8000 EU | 6.67 | 6.85 | 2.6% |
| 29 - 8000 EU | 7.26 | 6.73 | 7.6% |
| 30 - 8000 EU | 6.34 | 7.14 | 11.9% |
| 31 - 8000 EU | | 6.50 | |
| 32 - 8000 EU | | 6.61 | |
| 33 - 8000 EU | | 6.15 | |
| 34 - 8000 EU | | 5.87 | |
| 35 - 8000 EU | 5.43 | 6.31 | |
| 36 - 8000 EU | 6.57 | 6.40 | 2.6% |
| 37 - 8000 EU | | 7.50 | |
| 38 - 8000 EU | 7.18 | 7.51 | 4.5% |
| 39 - 8000 EU | 6.81 | 7.21 | 5.7% |
| 40 - 8000 EU | | 7.50 | |
| 41 - 8000 EU | 6.51 | 6.25 | 4.1% |
| 42 - 8000 EU | | 6.37 | |
| 43 - 8000 EU | 5.49 | 4.90 | |
| 44 - 8000 EU | | 4.83 | |
| 45 - 8000 EU | 5.48 | 5.09 | 7.4% |
| 46 - 8000 EU | | 5.03 | |
| 47 - 8000 EU | | 5.05 | |
| 48 - 8000 EU | | 5.60 | |
| 49 - 8000 EU | 7.09 | 6.59 | 7.3% |
| 50 - 8000 EU | | 5.30 | |
| Average ppm | 6.70 | 6.59 | 5.4% |

TABLE 3

| | Total (8) PAH (ppm) | | |
|---|---|---|---|
| | Industry | Exemplary Extraction Process | |
| Sample | Standard | Described Herein | RSD |
| 1 - DAE 70 | 117 | 128 | 9.0% |
| 2 - DAE 148 | 327 | 336 | 2.7% |
| 3 - DAE 250 | 247 | 267 | 7.8% |
| 4 - DAE 560 | 72.4 | 71.0 | 2.0% |

The industry standard method used for comparison is performed in accordance with EN 16143:2013 as follows:

General. Since the extender oils under investigation have a complex matrix which can hide the PAHs of interest, samples shall be submitted to a clean-up procedure before GC separation. The first cleaning step is done with Silica gel. For quantification a second cleaning step shall be performed on Sephadex LH20 or equivalent. These procedures are strongly recommended in order to make sure the samples are cleaned thoroughly before analyzing them by GC-MS.

### First Clean-Up Step Using Silica Column.

Preparation of the silica column. Before use, the silica gel is deactivated over 24 h by stirring with 7% (m/m) water which does not contain PAHs. Of the deactivated silica, 5 g is mixed with n-pentane and then transferred into a glass column Vibrate or knock mildly on the column to ensure that the silica is evenly and sufficiently packed without voids.

Chromatography on silica column. Run 10 ml of n-pentane through the column and discard the eluted volume. Apply the sample just before the last free n-pentane is about to vanish from the silica surface. Apply the sample in portions with a suitable Pasteur pipette. After the applied sample volume has vanished from the surface, apply an additional 2 ml portion of n-pentane to assure that whole sample is applied in the column. Just before the last n-pentane has vanished from the silica surface, apply 25 ml n-pentane in several portions. All volume eluted with n-pentane shall be discarded. Apply 75 ml cyclohexane in several portions and collect the eluted volume in a clean 100 ml volumetric flask. Concentrate the collected cyclohexane fraction using the sample concentrator to less than 1 ml, ensuring that the temperature does not exceed 35° C. If the second clean-up step is applied for quantitative results, this concentrate is to be submitted to further processing in the Sephadex clean-up step. If the Sephadex clean-up step is not used, transfer the concentrate to a 1 ml volumetric flask, add the injection standard to it and adjust the sample solution volume to 1 ml with Cyclohexane by filling up to the mark. This 1 ml sample is used to inject test portions for GC separation.

### Second Clean-Up Step Using Sephadex-LH 20 Column

Preparation of the Sephadex-LH20 column. Prepare a slurry of 5 g of dry Sephadex-LH20 and propanol-2, using a 3:1 (m/m) proportion for the solvent/Sephadex mixture. Let the slurry stand overnight for deactivation. Fill a glass column with 5 g of the deactivated slurry and drain the column from any free propanol-2, then close the bottom valve. When a fresh Sephadex-LH20 quality batch is used, it shall be tested with a solution of approximately 100 mg/l fluoranthene in propanol-2, so that it has an elution volume between 24 ml and 32 ml propanol-2 and that a straight level zone can be seen under UV-light (366 nm).

Chromatography on Sephadex-LH20 column. 2 ml to 3 ml of propanol-2 is added to the concentrate from above and evaporated next to dryness. The still damp residue is dissolved in 1 ml of propanol-2 and transferred to the Sephadex column with opened bottom valve using a suitable Pasteur pipette. Rinse the glass with 1 ml propanol-2 and transfer to the column. Continue adding propanol-2 so that the elution rate does not exceed 1 ml/min. The first collected 24 ml fraction may be discarded. The fraction from 24 ml to 70 ml contains all PAHs of interest, including the internal standards. Concentrate this fraction next to dryness at 35° C. The residue is then dissolved in 2 ml of acetone and transferred completely from the volumetric flask to a conical bottom flask, using an additional 1 ml of acetone for rinsing. Acetone is then carefully evaporated next to dryness. Dissolve the residue with 0.5 ml of cyclohexane, transfer to a 1 ml volumetric flask and rinse the glass with 0.3 ml cyclohexane and transfer again to the volumetric flask. Add the Injection standard to the flask and make up to 1 ml with cyclohexane.

In Tables 2 and 3 above and in Table 5, RSD is relative standard deviation. The results clearly show that the methods and compositions described herein are capable of closely replicating the extraction and analysis of PAHs using industry standard procedures.

In preferred embodiments of the methods described herein, the clean-up procedure is as follows:

Remove SPE cartridges from the bag. Add an extra layer C-18 to the SPE tube if desired. Place SPE cartridges on vacuum manifold. Flush with 20 mL of acetone. Tubes are now ready to use. Discard the acetone. Place the sample collection tubes into the vacuum manifold rack. Pour each sample into its SPE tube. Collect the clean sample, The sample is ready to blow dry, reconstitute in toluene and analyze by GC-MS or GC-MS/MS.

Table 4 below shows the results of a series of extractions using the methods described herein, from asphalt pitch samples. The asphalt pitch was first softened with toluene and dried to a honey like consistency to facilitate solubility in pentane followed by an 80:20 acetonitrile—ethyl acetate mixture.

TABLE 4

| | Injection | | |
|---|---|---|---|
| Sample Name | Date/Time | Total EU8 PAH | |
| Asphalt Pitch-A | Jul. 9, 2024 17:58 | 8.924 | ppm |
| Asphalt Pitch-B | Jul. 9, 2024 18:58 | 8.613 | ppm |
| Asphalt Pitch-A | Jul. 11, 2024 19:39 | 8.942 | ppm |
| Asphalt Pitch-B | Jul. 11, 2024 20:39 | 8.690 | ppm |
| | Average | 8.8 | ppm |
| | Standard Deviation | 0.17 | ppm |
| | % RSD | 1.9% | |

Additional tests confirmed and validated the methods described herein. A series of oil samples—including samples of extender oils, precursor oils, and crude—were spiked with a known amount of PAH standard, extracted and analyzed to quantify the amount of each of the eight (8) PAHs found in each. Specifically, each sample was also spiked with an internal standard and 1 ppm of each of the PAHs. The original sample results were subtracted from the spiked sample results for each of the eight PAHs. The average percent recovery for each of the eight PAHs (using an internal standard) ranged from 91% to 101% of the 1 ppm, with RSD between 6.6% and 13.8%, well within 20%. Results are shown in Table 5 below. In Table 5, RSD is relative standard deviation. The results clearly show that the methods and compositions described herein are capable of accurately and precisely recovering a 1 ppm PAH spike, passing the standard validation test of RSD<20%, spike recovery between 80% and 120%.

TABLE 5

| | PAH Analysis* (ppm) | | | | | | | |
|---|---|---|---|---|---|---|---|---|
| Sample | 1 | 2 | 3 | 4 | 5 | 6 | 7 | 8 |
| Sundex 8000 EU | 0.861 | 1.041 | 0.906 | 0.899 | 0.854 | 1.139 | 0.893 | 0.880 |
| | 0.874 | 1.126 | 0.989 | 0.919 | 0.901 | 1.320 | 0.902 | 0.921 |
| | 0.892 | 0.863 | 0.868 | 0.817 | 0.860 | 0.940 | 0.822 | 0.886 |
| | 0.937 | 0.942 | 0.930 | 0.868 | 0.891 | 1.169 | 0.926 | 0.927 |
| | 0.960 | 0.975 | 0.869 | 0.831 | 0.881 | 0.962 | 0.954 | 0.898 |
| | 0.886 | 0.972 | 0.854 | 0.927 | 1.065 | 1.078 | 0.962 | 0.861 |
| | 0.868 | 0.883 | 0.917 | 0.992 | 1.104 | 1.015 | 0.878 | 0.846 |
| | 0.889 | 0.914 | 0.953 | 0.952 | 1.124 | 1.057 | 0.901 | 0.885 |
| Precursor Oils | 0.936 | 0.941 | 0.918 | 1.000 | 0.968 | 0.967 | 0.934 | 0.962 |
| E30, E31 Combined | 0.964 | 0.988 | 0.944 | 1.025 | 0.990 | 0.983 | 0.945 | 0.943 |
| | 1.024 | 1.054 | 1.007 | 1.059 | 1.059 | 1.217 | 1.007 | 1.022 |
| | 0.813 | 0.797 | 0.751 | 0.788 | 0.795 | 0.695 | 0.808 | 0.862 |
| | 0.940 | 0.937 | 0.888 | 0.896 | 0.903 | 0.904 | 0.939 | 0.996 |
| | 0.966 | 0.998 | 0.914 | 0.907 | 0.928 | 1.005 | 1.084 | 1.034 |
| | 0.879 | 0.880 | 0.815 | 0.830 | 0.811 | 0.972 | 0.871 | 0.881 |
| | 0.970 | 0.988 | 0.926 | 0.890 | 0.883 | 1.019 | 0.932 | 0.998 |
| | 0.934 | 0.952 | 0.897 | 0.888 | 0.882 | 0.980 | 0.914 | 0.994 |
| Crude Oil | 1.048 | 0.738 | 0.955 | 0.937 | 0.823 | 0.964 | 1.175 | 0.915 |
| TK2 | 1.013 | 0.671 | 1.038 | 1.078 | 0.922 | 0.819 | 1.150 | 1.030 |
| Accuracy: | 93% | 93% | 91% | 92% | 93% | 101% | 95% | 93% |
| Average % | | | | | | | | |
| Recovery | | | | | | | | |
| (using internal | | | | | | | | |
| standard) | | | | | | | | |
| Standard Deviation | 0.06 | 0.11 | 0.07 | 0.08 | 0.10 | 0.14 | 0.10 | 0.06 |
| Precision: | 6.6% | 11.7% | 7.3% | 8.8% | 10.5% | 13.8% | 10.3% | 6.7% |
| % RSD | | | | | | | | |

*1 = Benz(a)anthracene, 2 = Chrysene, 3 = Benzo(b)fluoranthene, 4 = Benzo(k)fluoranthene, 5 = Benzo(j)fluoranthene, 6 = Benz[e]pyrene, 7 = Benz[a]pyrene, 8 = Dibenz(a,h)anthracene

What is claimed is:

1. A method for preparing an extract of polycyclic aromatic hydrocarbons from a petroleum product, comprising:
mixing the petroleum product with an organic solvent to produce a solvent petroleum mixture;
mixing the solvent petroleum mixture with an extraction solvent mixture, wherein the extraction solvent mixture consists of acetonitrile and ethyl acetate in a volume ratio of acetonitrile to ethyl acetate of 70:30 to 90:10, to produce an oil residue and an extract; and
separating the oil residue from the extract, wherein the extract contains the polycyclic aromatic hydrocarbons.

2. The method of claim 1, wherein the organic solvent is selected from the group consisting of pentanes, hexanes, heptanes, and octanes.

3. The method of claim 1, wherein the organic solvent is pentane, isooctane, heptane, or cyclohexane.

4. The method of claim 1, wherein the volume ratio of acetonitrile to ethyl acetate is 80:20.

5. The method of claim 1, further comprising the step of transferring the extract to a polypropylene solid phase extraction tube for further processing before analysis or identification of the polycyclic aromatic hydrocarbons.

6. The method of claim 1, wherein the further processing comprises sample clean-up followed by solvent blow down and re-constitution in toluene.

7. The method of claim 1, wherein the petroleum product is a refined aromatic oil, a refined paraffinic oil, a crude oil distillation cut, a crude oil, or asphalt pitch.

8. A kit for analyzing an extract of polycyclic aromatic hydrocarbons from a petroleum product, comprising:
an organic solvent, for mixing with the petroleum product to produce a solvent petroleum mixture;
a solvent mixture that consists of a volume ratio of acetonitrile to ethyl acetate of 70:30 to 90:10, for mixing with the solvent petroleum mixture to produce a sample; and
a polypropylene solid phase extraction tube for receiving the sample for further processing and analysis.

9. The kit of claim 8, wherein the organic solvent is selected from the group consisting of pentanes, hexanes, heptanes, and octanes.

10. The kit of claim 8, wherein the organic solvent is pentane, isooctane, heptane, or cyclohexane.

11. The kit of claim 8, wherein the volume ratio of acetonitrile to ethyl acetate is 80:20.
